(19) United States
(12) Patent Application Publication
(10) Pub. No.: US 2024/0207228 A1
(43) Pub. Date: Jun. 27, 2024

(54) A COMBINATION COMPRISING A SPECIFIC HDAC6 INHIBITOR AND AT LEAST ONE CTLA4 CHECKPOINT INHIBITOR

(71) Applicant: ITALFARMACO SPA, Milano (IT)

(72) Inventors: Gianluca FOSSATI, Milano (IT); Flavio LEONI, Milano (IT); Pietro Samuele POZZI, Legnano (IT); Elisabetta GALBIATI, Monza (IT); Christian STEINKÜHLER, Roma (IT)

(21) Appl. No.: 18/555,103

(22) PCT Filed: Apr. 19, 2022
(86) PCT No.: PCT/EP2022/060287

§ 371 Date: Oct. 12, 2023

(30) Foreign Application Priority Data
Apr. 20, 2021 (IT) 102021000009926

(51) Int. Cl. A61K 31/41; A61K 39/00; A61K 39/395; A61P 35/00

(52) U.S. Cl. CPC A61K 31/41; A61K 39/3955; A61P 35/00; A61K 2039/545

(57) ABSTRACT

The present invention relates to a combination comprising N-hydroxy-4-((5-(thiophen-2-yl)-1H-tetrazol-1-yl)methyl)benzamide or a pharmaceutically acceptable salt thereof and at least one CTLA4 checkpoint inhibitor, useful in the immunotherapy of tumors and in the treatment of one or more HDAC6-mediated diseases.

### TECHNICAL FIELD

The present invention relates to a combination comprising N-hydroxy-4-((5-(thiophen-2-yl)-1H-tetrazol-1-yl)methyl)benzamide or a pharmaceutically acceptable salt thereof and at least one CTLA4 checkpoint inhibitor, useful in the immunotherapy of tumors and in the treatment of one or more diseases HDAC6-mediated.

### BACKGROUND

N-hydroxy-4-((5-(thiophen-2-yl)-1H-tetrazol-1-yl)methyl)benzamide (herein indicated also as ITF3756) is the compound 8 disclosed in WO2018/189340, that also discloses its method of synthesis and its activity as HDAC6 inhibitor and in the treatment of graft rejection, GVHD, myositis, diseases associated with abnormal lymphocyte function, multiple myeloma, non-Hodgkin lymphoma, peripheral neuropathy, autoimmune diseases, inflammatory diseases, cancer and neurodegenerative pathologies.

The human HDAC class consists of 18 enzymes, divided into two groups: zinc-dependent HDACs and NAD-dependent HDACs, also known as sirtuins (class III). Zinc-dependent HDACs are further distributed into four classes: 1) Class I, including HDAC1, 2, 3 and 8, ubiquitous isoenzymes mainly located in the nucleus; 2) Class IIa, including HDAC4, 5, 7 and 9, isoenzymes located both in the nucleus and the cytoplasm; 3) Class IIb, including HDAC6 and HDAC10, mainly located in the cytoplasm and 4) Class IV, including only HDAC11. Unlike Class I HDACs, Class IIa and IIb have a tissue-specific expression.

Selective inhibitors for an HDAC family or for a specific isoform, especially HDAC6, may be particularly useful for treating pathologies related to proliferative disorders and protein accumulation, immune system disorders and neurological and neurodegenerative disease, such as stroke, Huntington's disease, ALS and Alzheimer's disease.

Particularly for the HDAC6 isoform, different substrates have been identified, such as α-tubulin, Hsp90 (Heat Shock Protein 90), cortactin, β-catenin. Modulation of these proteins acetylation by HDAC6 has been correlated with several important processes, such as immune response (J. Med. Chem. (2012), 55, 639-651; Mol. Cell. Biol. (2011), 31(10), 2066-2078), regulation of microtubule dynamics, including cell migration and cell-cell interaction (Aldana-Masangkay et al., J. Biomed. Biotechnol. (2011), 2011, 875824), and degradation of degenerated proteins.

In addition, HDAC6 is involved in the process of catabolism of degraded proteins through the complex known as aggresome: HDAC6 is able to bind polyubiquitinated proteins and dynein, thus activating a kind of delivery of denatured proteins along the microtubules to the aggresome (Kawaguchi et al., Cell (2003) 115 (6), 727-738).

Alteration of this HDAC6 cytoprotective activity has been correlated with various neurodegenerative pathologies such as Parkinson's disease (Outerio et al., Science (2007), 317 (5837), 516-519) and Huntington's disease (Dompierre et al., J. Neurosci. (2007), 27(13), 3571-3583), wherein the accumulation of degraded proteins is a common pathological feature.

Furthermore, HDAC6 is involved in regulating many oncological proteins, especially in hematologic tumours, such as various types of leukaemia (Fiskus et al., Blood (2008), 112(7), 2896-2905) and multiple myeloma (Hideshima et al., Proc. Natl. Acad. Sci. USA (2005), 102(24), 8567-8572). Regulation of α-tubulin acetylation by HDAC6 may be implicated in metastasis onset, wherein cellular motility plays an important role (Sakamoto et al., J. Biomed. Biotechnol. (2011), 2011, 875824). Recently, HDAC6 has attracted interest as a novel immune-oncology target, due to the fact that this enzyme was shown to be an obligate regulator of the expression of the immune-checkpoint protein PD-L1 (Lienlaf et al. Mol Oncol 2016 May; 10(5):735-750). HDAC6 inhibitors were shown to be effective in preclinical immuno-oncology models and to enhance the activity of anti PD-1 antibodies (Ray et al., Leukemia 2018 March; 32(3):843-846. Keremu et al Cancer Chemother Pharmacol. 2019 February; 83(2):255-264. Knox et al. Sci Rep. 2019 Apr. 16; 9(1):6136).

CTLA4 or CTLA-4 (cytotoxic T-lymphocyte-associated protein 4) is a protein receptor that functions as an immune checkpoint and downregulates immune responses. CTLA4 is constitutively expressed in regulatory T cells but only upregulated in conventional T cells after activation—a phenomenon which is particularly notable in cancers. It acts as an “off” switch when bound to CD80 or CD86 on the surface of antigen-presenting cells.

There is increasing interest in the possible therapeutic benefits of blocking CTLA4 (using antagonistic antibodies against CTLA4). Ipilimumab was the first anti-CTLA4 antibody approved by U.S. Food and Drug Administration (FDA) in March 2011, for the treatment of melanoma. FDA has also approved the anti-CTLA4 therapy ipilimumab 3 mg/kg plus the anti-PD-1 therapy nivolumab 1 mg/kg for patients with advanced melanoma. This regimen has been shown to increase survival, delay progression, and increase the proportion of patients achieving an objective response compared with ipilimumab alone (Larkin J et al. n engl j med 381; 16 2019). However, these benefits came with increased toxicity, including a high proportion of grade 3/4 treatment-related adverse events. For example, the treatment with nivolumab is associated to the following adverse events: nephritis, hepatitis, pancreatitis, and pneumonitis (Kang J H et al. Trends in Immunology 42, 293, 2021).

A lot of interest has been shown in combining checkpoint inhibitors with other agents. The goal, of course, is to find the right other agent. In the world of lung cancer and now breast cancer, and other diseases in which chemotherapy is effective, researchers are looking at the combination chemotherapy with immune checkpoint inhibitor therapy, which has produced some success.

However, some failures have occurred such as the Phase III trial of pembrolizumab (anti PD-1) and the indoleamine 2,3-dioxygenase 1 (IDO1) inhibitor epacadostat in subjects with melanoma that was halted in April 2018 as the combination therapy missed the first primary endpoint of improving progression-free survival vs. pembrolizumab alone.

From a mechanistic standpoint, it made sense that this combination would produce good results, however, the spectacular failure of this combination provides an excellent lesson in how the researchers need to do a better job of vetting the combinations that move forward in development.

### SUMMARY OF THE INVENTION

An object of the present invention is therefore to provide a new combination which maintains or improves the therapeutic efficacy of the known and approved combinations and/or of the drugs administered individually but which has, at the same time, a better toxicological profile.

The present inventors have surprisingly found that the combination comprising ITF3756 and the anti-CTLA4 antibody shows an anti-tumor effect superior to the administration of the single drugs and said combination shows a synergistic therapeutic effect. In particular, the most potent anti-tumor effect was obtained with the administration of ITF3756 50 mg/Kg three times a day in combination with anti-CTLA4 10 mg/kg, which would prevent also the development of secondary tumors.

The present inventors have also surprisingly found that the combination treatment according to the present invention could have a better safety profile than the combination anti-CTLA4 and anti-PD1 or the combination anti-CTLA4 and anti-PD-L1, while maintaining the same efficacy on tumor growth inhibition.

Accordingly, a first object of the present invention is a combination comprising N-hydroxy-4-((5-(thiophen-2-yl)-1H-tetrazol-1-yl)methyl)benzamide or a pharmaceutically acceptable salt thereof, and at least one CTLA4 checkpoint inhibitor.

The second object of the present invention is said combination for use as a medicament.

### DESCRIPTION OF THE FIGURES

FIG. 1 shows effect of ITF3756 treatment on CT26 tumor growth in the mouse.

FIG. 2 shows effect of anti-CTLA4 Mab on CT26 tumor growth in the mouse.

FIG. 3 shows effects ITF3756, at the dose of 25 mg/Kg in combination with anti-CTLA4 Mab, on CT26 tumor growth in the mouse.

FIG. 4 shows effect of ITF3756, at the dose of 50 mg/Kg in combination with anti-CTLA4 Mab, on CT26 tumor growth in the mouse.

FIG. 5 shows effect of ITF3756, at the dose of 50 mg/Kg×3 in combination with anti-CTLA4 Mab, on CT26 tumor growth in the mouse.

FIG. 6 shows effect of prolonged administration of ITF3756, at the dose of 50 mg/Kg, three times a day and once a day, in combination with anti-CTLA4 Mab, on CT26 tumor growth in the mouse.

FIG. 7 shows effect of previous administration of ITF3756 in combination with anti-CTLA4 on the growth of secondary tumors (tumor challenge) in the CT26 murine model.

FIGS. 8A-8B show effect of previous administration of ITF3756 (50 mg/Kg once a day) in combination with anti-CTLA4 (10 mg/kg) on the growth of secondary tumors (tumor challenge) in the CT26 murine model.

FIGS. 9A-9B show effect of previous administration of ITF3756 (50 mg/Kg three times a day) in combination with anti-CTLA4 (10 mg/kg) on the growth of secondary tumors in the CT26 murine model.

FIG. 10 shows effect of ITF3756 in combination with anti-CTLA4 to prevent tumor growth vs. the combination anti PD-1+anti-CTLA4.

FIG. 11 shows effect of ITF3756 alone or in combination with anti-CTLA4 on diabetes in female NOD mice.

### DEFINITIONS

Unless otherwise defined, all terms of art, notations and other scientific terminology used herein are intended to have the meanings commonly understood by those of skill in the art to which this disclosure pertains. In some cases, terms with commonly understood meanings are defined herein for clarity and/or for ready reference; thus, the inclusion of such definitions herein should not be construed to represent a substantial difference over what is generally understood in the art.

The term “physiologically acceptable excipient” herein refers to a substance devoid of any pharmacological effect of its own and which does not produce adverse reactions when administered to a mammal, preferably a human. Physiologically acceptable excipients are well known in the art and are disclosed, for instance in the Handbook of Pharmaceutical Excipients, sixth edition 2009, herein incorporated by reference.

The term “Pharmaceutically acceptable salts” herein refers to those salts which possess the biological effectiveness and properties of the salified compound and which do not produce adverse reactions when administered to a mammal, preferably a human. The pharmaceutically acceptable salts may be inorganic or organic salts; examples of pharmaceutically acceptable salts include but are not limited to: carbonate, hydrochloride, hydrobromide, sulphate, hydrogen sulphate, citrate, maleate, fumarate, trifluoroacetate, 2-naphthalenesulphonate, and para-toluenesulphonate. Further information on pharmaceutically acceptable salts can be found in Handbook of pharmaceutical salts, P. Stahl, C. Wermuth, WILEY-VCH, 127-133, 2008, herein incorporated by reference.

The term “simultaneous, separate or sequential administration” herein refers to administration of the first and second compound at the same time or in such a manner that the two compounds act in the patient's body at the same time or administration of one compound after the other compound in such a manner to provide a therapeutic effect. In some embodiments the compounds are taken with a meal. In other embodiments, the compounds are taken after a meal, such as 30 minutes or 60 minutes after a meal. In some embodiments, one compound is administered to a patient for a time period followed by administration of the other compound.

The term “CTLA4 checkpoint inhibitor” or “Anti-CTLA4” or “Anti-CTLA4 antibody” according to the present application refers to any compound able to inhibit, partially or totally, the biological activity of CTLA4 (cytotoxic T-lymphocyte-associated protein 4) immune checkpoint.

The terms “approximately” and “about” herein refers to the range of the experimental error, which may occur in a measurement.

The terms “comprising”, “having”, “including” and “containing” are to be construed open-ended terms (i.e. meaning “including, but not limited to”) and are to be considered as providing support also for terms as “consist essentially of”, “consisting essentially of”, “consist of” or “consisting of”.

The terms “consist essentially of”, “consisting essentially of” are to be construed as semi-closed terms, meaning that no other ingredients which materially affects the basic and novel characteristics of the invention are included (optional excipients may thus included).

The terms “consists of”, “consisting of” are to be construed as closed terms.

### DETAILED DESCRIPTION OF THE INVENTION

As it will be disclosed in details in the Experimental Section, the present inventors have found that the treatment combination of ITF3756 (25 mg/Kg and 50 mg/Kg)+anti-CTLA (10 mg/Kg) resulted more active than the administration of the single drugs.

In particular, the data obtained demonstrate the synergistic effect of the combined ITF3756 and anti-CTLA4 with respect to ITF3756 and anti-CTLA4 administered alone.

Furthermore, the present inventors have also found that the most potent anti-tumor effect was obtained with the administration of ITF3756 (50 mg/Kg three times a day) in combination with anti-CTLA4 (10 mg/kg).

The results obtained suggest an activation of the immune system induced by the treatment combination. In fact, the combination of the present invention would prevent the development of secondary tumors.

The inventors have further found that ITF3756 decreases cytokine-induced expression of PD-L1 in human monocytes stimulated in vitro and that the administration of ITF3756 (50 mg/Kg three times a day) decreased PD-L1 expression in mouse immune cells in vivo. Treatment of NOD mice with anti PD-1, anti PD-L1 antibodies or the with combination of anti PD-1 or anti PD-L1 antibodies with an anti CTLA4 antibody potently accelerates the induction of auto-immune diabetes, a known side-effect of clinical relevance. Surprisingly, the inventors found that treatment of NOD mice with ITF3756 (50 mg/Kg three times a day) either alone or in combination with an anti CTLA4 antibody did not accelerates the induction of autoimmune diabetes, suggesting that this combination treatment is much better tolerated and will give rise to less auto-immune adverse events when compared to the combination of anti PD(L)1 and anti CTLA4 antibodies.

Accordingly, a first object of the present invention is a combination comprising N-hydroxy-4-((5-(thiophen-2-yl)-1H-tetrazol-1-yl)methyl)benzamide or a pharmaceutically acceptable salt thereof, and at least one CTLA4 checkpoint inhibitor.

According to a preferred embodiment, said at least one CTLA4 checkpoint inhibitor is selected from ipilimumab or tremelimumab.

The second object of the present invention is the combination according to the invention for use as a medicament.

Preferably, the combination is useful in the treatment of any disease or condition susceptible of being improved or prevented by treatment with anti CTLA4, anti PD1 and/or anti PD-L1 antibodies.

According to a preferred embodiment of the present invention, the combination is useful in the treatment of a patient who has discontinued treatment with anti CTLA4, anti PD1 and/or anti PDL1 antibodies. In particular, the patient has discontinued treatment with anti CTLA4, anti PD1 and/or anti PDL1 antibodies because of toxicity.

According to a preferred embodiment of the present invention, the combination is useful in the treatment of a patient who is not treated with anti CTLA4, anti PD1 and/or anti PDL1 antibodies. In particular, the patient is not treated with anti CTLA4, anti PD1 and/or anti PDL1 antibodies because of expected toxicity.

The combination of the invention is preferably useful for the immunotherapy of tumors and the treatment of HDAC6-mediated diseases.

According to a preferred embodiment of the present invention, the combination is useful in the treatment of one or more diseases selected from the group: Adrenocortical Carcinoma, Anal Cancer, Astrocytomas, Basal Cell Carcinoma of the Skin, Bladder Cancer, Brain Tumors, Breast Cancer, Carcinoma of Unknown Primary, Cardiac Tumors, Cervical Cancer, Cholangiocarcinoma, Colorectal Cancer, Endometrial Cancer, Esophageal Cancer, Intraocular Melanoma, Fallopian Tube Cancer, Gallbladder Cancer, Gastric Cancer, Gastrointestinal Carcinoid Tumor, Gastrointestinal Stromal Tumors (GIST), Germ Cell Tumors, Testicular Cancer, Head and Neck Cancer, Hepatocellular Carcinoma, Islet Cell Tumors, Pancreatic Neuroendocrine Tumors, Langerhans Cell Histiocytosis, Leukemias, Lung Cancer (Non-Small Cell, Small Cell, Pleuropulmonary Blastoma, and Tracheobronchial Tumor), Melanoma, Merkel Cell Carcinoma, Mesothelioma, Midline Tract Carcinoma With NUT Gene Changes, Multiple Endocrine Neoplasia Syndromes, Multiple Myeloma/Plasma Cell Neoplasms, Myelodysplastic Syndromes, Myelodysplastic/Myeloproliferative Neoplasms, Neuroblastoma, Ovarian Cancer, Pancreatic Cancer, Paraganglioma, Parathyroid Cancer, Penile Cancer, Pheochromocytoma, Pituitary Tumor, Primary Peritoneal Cancer, Prostate Cancer, Renal Cell Cancer, Retinoblastoma, Sarcomas, Squamous Cell Carcinoma of the Skin, Thymoma and Thymic Carcinoma, Thyroid Cancer, Transitional Cell Cancer of the Renal Pelvis and Ureter, Uterine Cancer, Vaginal Cancer, Vascular Tumors, Vulvar Cancer, Wilms Tumor. Preferably, the combination is useful in the treatment of melanoma, breast cancer, renal cell carcinoma, non small cell lung cancer and colorectal cancer.

According to a preferred embodiment, the combination for use according to the present invention is characterized in that N-hydroxy-4-((5-(thiophen-2-yl)-1H-tetrazol-1-yl)methyl)benzamide or a pharmaceutically acceptable salt thereof and said at least one CTLA4 checkpoint inhibitor are for simultaneous, separate or sequential administration.

Preferably, N-hydroxy-4-((5-(thiophen-2-yl)-1H-tetrazol-1-yl)methyl)benzamide or a pharmaceutically acceptable salt thereof is administered to a patient on a daily basis, preferably from 2 to three times a day and said at least one CTLA4 checkpoint inhibitor is administered to a patient every 2 to 4 weeks, preferably for a maximum of 4 doses.

Preferably, N-hydroxy-4-((5-(thiophen-2-yl)-1H-tetrazol-1-yl)methyl)benzamide or a pharmaceutically acceptable salt thereof is administered to a patient by oral route.

Preferably, the CTLA4 checkpoint inhibitor is administered by intravenous infusion. More preferably, N-hydroxy-4-((5-(thiophen-2-yl)-1H-tetrazol-1-yl)methyl)benzamide or a pharmaceutically acceptable salt thereof is administered to a patient in an amount ranging from 200 mg to 1000 mg BID or from 100 mg to 1000 mg TID and said at least one CTLA4 checkpoint inhibitor is administered to a patient in an amount ranging from 0.5 to 10 mg/kg every 2 to 4 weeks, preferably from 1 to 3 mg/kg every 2 to 4 weeks.

The human doses for ITF3756 were predicted using a physiologically based pharmacokinetic (PBPK) model, through the use of the software GastroPlus™ (Simulation Plus, Lancaster, CA). The target C_{average} for projected doses was 200 ng/mL, mean level in 24 hours found after the administration schedule that had shown efficacy in the mouse.

The modelling strategy was to establish and evaluate models first for animal species for which there was experimental in vivo pharmacokinetics data available (mouse, rat, dog and Cynomolgus monkey) and thereafter utilize species specific input and physiological data with consistent in vitro to in vivo scaling approaches for projection of human pharmacokinetics. The same work was done on ITF2357 (givinostat), a compound with existing clinical data, with similarities in elimination mechanism with ITF3756, to support assumptions made for elimination of ITF3756.

The model was built using physicochemical characteristics and in vitro data of ITF3756, such as Log D, pkA, solubility in water and in biorelevant fluids, permeability, and protein binding and blood to plasma partitioning generated for each species.

Hepatic metabolism was studied starting from data generated in vitro after incubation of ITF3756 with species specific cryopreserved hepatocytes. Renal and intestinal metabolism were deduced from in vitro data collected after incubation of the test item with species specific kidney and intestine microsomes with NADPH and UDPGA as cofactors. In vitro to in vivo extrapolations were then made using opportune scaling factors. Whole blood clearance was used as a surrogate for extrahepatic metabolism. Tissue concentrations were predicted assuming all tissues to behave as well-stirred compartments with perfusion rate-limited distribution, and drug and tissue specific tissue to plasma partitioning coefficient Kp. Kp values were generally predicted from drug physicochemical properties and tissue composition.

Absorption and transit through the gut wall were predicted using the ACAT model (Advanced Compartmental Absorption and Transit), integrated in the whole body PBPK model.

In the first step pharmacokinetic parameters after intravenous administration were predicted in each animal species. Empirical scaling factors were used for extrahepatic metabolism to capture body clearance, and for Log D to capture volume of distribution. Based on empirical scaling factors, low and high clearance scenarios were found.

Pharmacokinetics after oral administration were then modelized, finding uncertainty in permeability, solubility and dissolution, due to interspecies variability. Eight scenarios were then outlined, among which worst and best case oral absorption scenarios could be found.

Projections of human pharmacokinetics were then made using the 8 scenarios, derived from the combination of best and worst case of bioavailability, high and low clearance and high and low volume of distribution. Depending on which of the simulated scenarios captures the human pharmacokinetics the best, the dosing regimen, predicted to reach the target exposure, range from 200 mg to 1000 mg BID or from 100 mg to 1000 mg TID as mentioned above.

The invention will be further illustrated in greater details in the following experimental section.

### EXPERIMENTAL SECTION

### Example 1

Anti-Tumor Effect of ITF3756 Administered in Combination with Anti-CTLA4 Antibody in the CT26 Murine Model

The anti-tumor efficacy of ITF3756, administered in combination with anti-CTLA4 antibody, has been determined using a murine model based on the use of the mouse colon carcinoma cell line CT26. In this model, the CT26 cells are implanted subcutaneously to syngeneic mice and the efficacy is determined as tumor growth inhibition on the basis of the volume of the tumor nodule.

### Materials and Methods

### ITF3756 (N-hydroxy-4-((5-(thiophen-2-yl)-1H-tetrazol-1-yl)methyl)benzamide)

ITF3756 was synthetized by the Medicinal Chemistry Dept. of Italfarmaco SpA. ITF3756, batch 5, as powder was solubilized in DMSO and stored at −20° C. On each day of administration, the solution was diluted with H₂O/PEG 400 1:1 to obtain a final solution of H₂O/PEG 400 1:1 in DMSO 0.5% at 2.5 and 5.0 mg/mL.

The solutions were administered orally by using gavage needles for mice, in a volume of 200 μL (final doses 25 and 50 mg/Kg).

ITF3756 administration started when the tumor nodules were palpable (around day 10 from cells inoculation). ITF3756 was administered orally (os) once a day or three times a day, as reported in Table 1.

### Anti-CTLA4 Antibody

Syrian hamster IgG Mab anti-mouse CTLA4 was purchased from BioXell (cat. BE0131, clone 9H10), diluted with PBS at 1, 0.3 and 0.1 mg/mL final concentration and stored at +4° C. Each mouse was treated with 200 μL of the respective solution (final doses 10.0, 3.0 and 1.0 mg/Kg).

Anti-CTLA4 Mab administration started when the tumor nodules were palpable (around day 10 from cells inoculation). Anti-CTLA4 was administered intraperitoneally (ip) once a day on alternate days for a total of 4 treatments, followed by 7 days of wash-out, as reported in table 1. This treatment cycle continued up to the end of the study.

### In Vivo Study

Female 6 weeks old BALB/c mice were purchased from Charles River Italia and maintained with food and water ad libitum, under light-dark cycle of 12 hours.

After 5 days of acclimatization, the mice were submitted to tumor cells injection.

CT26 (murine BALB/c colon carcinoma, CT26.WT ATCC CRL-2638) cells were grown in RPMI 1640 cell culture medium+10% fetal calf serum (FCS).

The cells were detached, during the exponential phase, with trypsin, washed with the culture medium without FCS and suspended at 5×10⁶ cells/mL final concentration.

The cells (1×10⁶ cells/mouse) were injected s.c. in the inguinal region of the right flank of the mice in a volume of 200 μL.

When the tumor nodule was palpable in at least 80% of the animals, the mice were randomized in the following experimental groups and drugs administration was started.

TABLE 1
Experimental groups and treatment schedules.

| Group Number | | |
|---|---|---|
| (animals/group) | Experimental group | Treatment schedule |
| 1 (n = 6) | Control, Vehicles* | Orally, once a day. |
| | | ip, 4 total admin. on alternate days, |
| | | followed by 7 days of wash-out |
| 2 (n = 3) | Control, Vehicles* | Orally, 3 times a day. |
| | | ip, 4 total admin. on alternate days, |
| | | followed by 7 days of wash-out |
| 3 (n = 3) | Control, untreated | — |
| 4 (n = 6) | ITF3756, 25 mg/Kg | Orally, once a day. |
| 5 (n = 6) | ITF3756, 50 mg/Kg | Orally, once a day. |
| 6 (n = 6) | ITF3756, 50 mg/Kg | Orally, 3 times a day. |
| 7 (n = 6) | Anti-CTLA4, 1 mg/Kg | ip, 4 total admin. on alternate days, |
| | | followed by 7 days of wash-out |
| 8 (n = 6) | Anti-CTLA4, 3 mg/Kg | ip, 4 total admin. on alternate days, |
| | | followed by 7 days of wash-out |
| 9 (n = 6) | Anti-CTLA4, 10 mg/Kg | ip, 4 total admin. on alternate days, |
| | | followed by 7 days of wash-out |
| 10 (n = 6) | ITF3756, 25 mg/Kg + | Orally, once a day. |
| | anti-CTLA4, 1 mg/Kg | ip, 4 total admin. on alternate days, |
| | | followed by 7 days of wash-out |
| 11 (n = 6) | ITF3756, 25 mg/Kg + | Orally, once a day. |
| | anti-CTLA4, 3 mg/Kg | ip, 4 total admin. on alternate days, |
| | | followed by 7 days of wash-out |
| 12 (n = 6) | ITF3756, 25 mg/Kg + | Orally, once a day. |
| | anti-CTLA4, 10 mg/Kg | ip, 4 total admin. on alternate days, |
| | | followed by 7 days of wash-out |
| 13 (n = 6) | ITF3756, 50 mg/Kg + | Orally, once a day. |
| | anti-CTLA4, 1 mg/Kg | ip, 4 total admin. on alternate days, |
| | | followed by 7 days of wash-out |
| 14 (n = 6) | ITF3756, 50 mg/Kg + | Orally, once a day. |
| | anti-CTLA4, 3 mg/Kg | ip, 4 total admin. on alternate days, |
| | | followed by 7 days of wash-out |
| 15 (n = 6) | ITF3756, 50 mg/Kg + | Orally, once a day. |
| | anti-CTLA4, 10 mg/Kg | ip, 4 total admin. on alternate days, |
| | | followed by 7 days of wash-out |
| 16 (n = 6) | ITF3756, 50 mg/Kg + | Orally, 3 times a day. |
| | anti-CTLA4, 10 mg/Kg | ip, 4 total admin. on alternate days, |
| | | followed by 7 days of wash-out |

*Control animals were treated orally with ITF3756 vehicle (H₂O/PEG 400 1:1 + DMSO 0.5%) and ip with anti-CTLA4 vehicle (PBS).

Body weight was measured on alternate days, starting from the first day of drug administration.

The volume of subcutaneous tumor nodules was determined on alternate days, according to the following formula (Papagiannoros A. et al. in vivo 20:129-136, 2006):

Volume (mm³)=(D×d²)/2

where D=major diameter of the nodule and d=minor diameter of the nodule.

The weight of the tumor nodule was determined by the tumor volume considering a tumor density of 1.05 g/mL (Jensen M. M. et al. BMC Medical Imaging 8(16), 2008).

The animals were euthanized when the tumor weight was equal to the 10% of body weight or the tumor nodules were ulcerated (humane end-points).

The statistical analysis of the drugs effect was carried out by 2-way ANOVA with Dunnett's multiple comparisons test using GraphPad Prism 8 software.

### Results

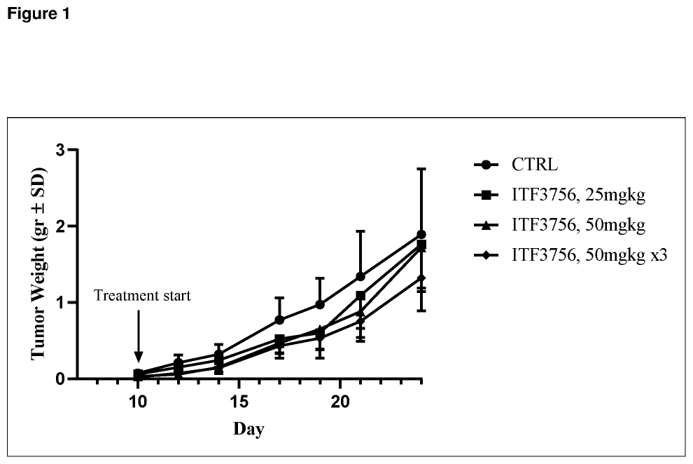

The effect of ITF3756 on the growth of CT26 tumor is summarized in FIG. 1.

The dose of 25 mg/Kg (once a day) did not show a significant inhibition of tumor growth throughout the entire experimental period. The higher dose (50 mg/Kg, once a day) exerted a significant effect on day 21 with 34% inhibition (0.88 g vs 1.34 g) of tumor growth that was no more detected on day 24 (9% inhibition).

On the contrary, the animals treated with ITF3756 50 mg/Kg 3 times a day, showed a significant reduction of tumor starting from day 19 up to day 24 (30% inhibition, 1.32 g vs 1.89 g) indicating that this schedule of treatment exerted a pharmacological effect superior to the single daily administration.

| | Statistical significance vs Control group on day | | | | | | |
|---|---|---|---|---|---|---|---|
| Group | 10 | 12 | 14 | 17 | 19 | 21 | 24 |
| ITF3756, | n.s. | n.s. | n.s. | n.s. | n.s. | n.s. | n.s. |
| 25 mg/Kg | | | | | | | |
| ITF3756, | n.s. | n.s. | n.s. | n.s. | n.s. | * | n.s. |
| 50 mg/Kg | | | | | | | |
| ITF3756, | n.s. | n.s. | n.s. | n.s. | * | ** | ** |
| 50 mg/Kg x3 | | | | | | | |

n.s. = not significant,
* p < 0.05,
** p < 0.01

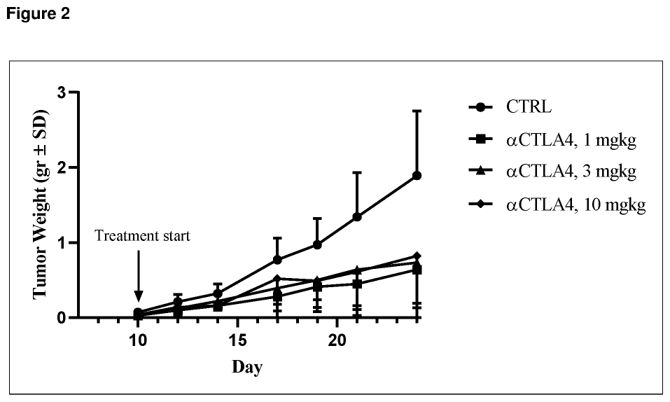

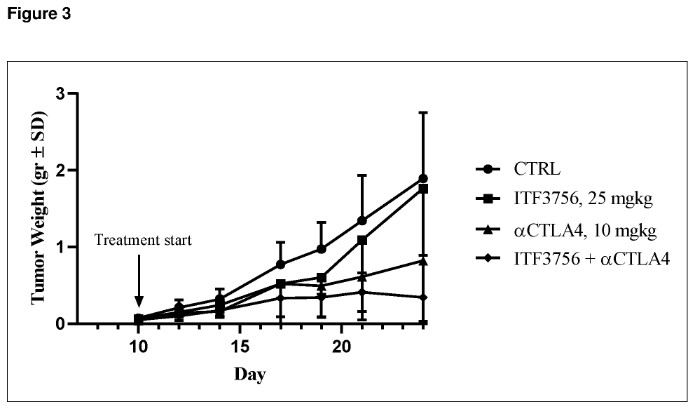

The effect of the anti-CTLA4 antibody is summarized in FIG. 2. All the 3 doses of antibody resulted similarly active with a maximal effect of tumor inhibition on day 24 (66-57%).

| | Statistical significance vs Control group on day | | | | | | |
|---|---|---|---|---|---|---|---|
| Group | 10 | 12 | 14 | 17 | 19 | 21 | 24 |
| αCTLA4 | n.s. | n.s. | n.s. | * | * | *** | *** |
| 1 mg/Kg | | | | | | | |
| αCTLA4 | n.s. | n.s. | n.s. | n.s. | n.s. | ** | *** |
| 3 mg/Kg | | | | | | | |
| αCTLA4 | n.s. | n.s. | n.s. | n.s. | * | *** | *** |
| 10 mg/Kg | | | | | | | |

n.s. = not significant,
* p < 0.05,
** p < 0.01,
*** p < 0.001

The effect of ITF3756 administered at 25 mg/Kg in combination with the highest dose Statistical significance vs Control group on day

| | Statistical significance vs Control group on day | | | | | | |
|---|---|---|---|---|---|---|---|
| Group | 10 | 12 | 14 | 17 | 19 | 21 | 24 |
| ITF3756 | n.s. | n.s. | n.s. | n.s. | n.s. | n.s. | n.s. |
| 25 mg/Kg | | | | | | | |
| αCTLA4 | n.s. | n.s. | n.s. | n.s. | * | ** | *** |
| 10 mg/Kg | | | | | | | |
| ITF3756 | n.s. | n.s. | n.s. | n.s. | * | *** | *** |
| 25 mg/Kg + | | | | | | | |
| αCTLA4 | | | | | | | |
| 10 mg/Kg | | | | | | | |

n.s. = not significant,
* p < 0.05,
** p < 0.01,
*** p < 0.001

The treatment combination of ITF3756 (25 mg/Kg)+anti-CTLA (10 mg/Kg) according to the present invention resulted more active than the administration of the single drugs. Moreover, said combination shows a synergistic therapeutic effect.

In fact, on day 24, the drugs combination induced an 82% inhibition (0.34 g vs 1.89 g) of tumor growth, whereas ITF3756 and anti-CTLA4 alone induced 7% (1.76 vs 1.89 g) and 57% inhibition (0.82 vs 1.89 g), respectively.

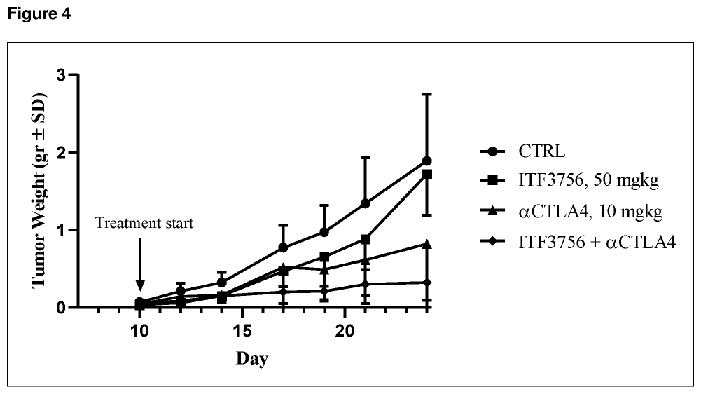

The effect of ITF3756 administered at 50 mg/Kg in combination with the highest dose (10 mg/Kg) of the anti-CTLA4 antibody is reported in FIG. 4.

| | Statistical significance vs Control group on day | | | | | | |
|---|---|---|---|---|---|---|---|
| Group | 10 | 12 | 14 | 17 | 19 | 21 | 24 |
| ITF3756 | n.s. | n.s. | n.s. | n.s. | n.s. | * | n.s. |
| 50 mg/Kg | | | | | | | |
| αCTLA4 | n.s. | n.s. | n.s. | n.s. | * | *** | *** |
| 10 mg/Kg | | | | | | | |
| ITF3756 | n.s. | n.s. | n.s. | ** | *** | *** | *** |
| 50 mg/Kg + | | | | | | | |
| αCTLA4 | | | | | | | |
| 10 mg/Kg | | | | | | | |

n.s. = not significant,
* p < 0.05,
** p < 0.01,
*** p < 0.001

The treatment combination of ITF3756 (50 mg/Kg)+anti-CTLA (10 mg/Kg) resulted more active than the administration of the single drugs. Moreover, said combination shows a synergistic therapeutic effect.

In fact, on day 24, the drugs combination induced an 83% inhibition (0.32 vs 1.89 g) of tumor growth, whereas ITF3756 and anti-CTLA4 alone induced 9% (1.72 vs 1.89 g) and 57% (0.82 vs 1.89 g) inhibition, respectively.

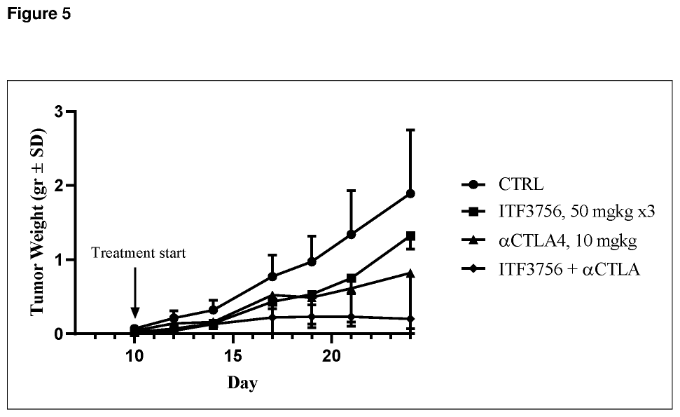

The effect of ITF3756 administered at 50 mg/Kg 3 times a day, in combination with the highest dose (10 mg/Kg) of the anti-CTLA4 antibody is reported in FIG. 5.

| | Statistical significance vs Control group on day | | | | | | |
|---|---|---|---|---|---|---|---|
| Group | 10 | 12 | 14 | 17 | 19 | 21 | 24 |
| ITF3756 | n.s. | n.s. | n.s. | n.s. | * | ** | ** |
| 50 mg/Kg x3 | | | | | | | |
| αCTLA4 | n.s. | n.s. | n.s. | n.s. | * | *** | *** |
| 10 mg/Kg | | | | | | | |
| ITF3756 | n.s. | n.s. | n.s. | * | *** | *** | *** |
| 50 mg/Kg x3 + | | | | | | | |
| αCTLA4 | | | | | | | |
| 10 mg/Kg | | | | | | | |

n.s. = not significant,
* p < 0.05,
** p < 0.01,
*** p < 0.001

The treatment combination of ITF3756 (50 mg/Kg×3)+anti-CTLA (10 mg/Kg) resulted more active than the administration of the single drug. Moreover, said combination shows a synergistic therapeutic effect.

In fact, on day 24, the drugs combination induced 89% (0.2 vs 1.89 g) inhibition of tumor growth, whereas ITF3756 and anti-CTLA4 alone induced 30% (1.32 vs 1.89 g) and 57% (0.82 vs 1.89 g) inhibition, respectively. Moreover, the efficacy of the combination treatment on day 24 resulted significantly higher than that exerted by the anti-CTLA4 antibody alone (p<0.001).

Since the tumors growth of the animals treated with both the drug combinations (anti-CTLA4+ITF3756 50 mg/Kg once a day and 3 times a day) was almost completely abolished on day 24 (last experimental day of the control group), the drug administration to these two groups was prolonged up to day 42.

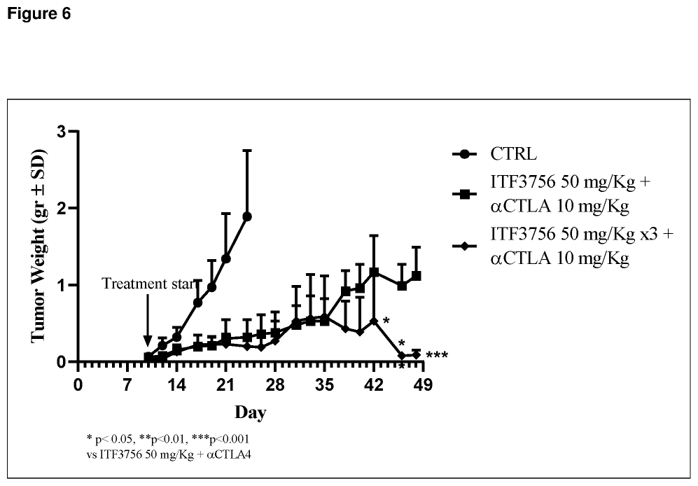

The results obtained are reported in FIG. 6. In the graph is reported the average volume of the tumors that were measurable.

Both drug combinations according to the present invention induced a superimposable and potent inhibition of tumor growth up to day 35. From this day on the tumors of the animals treated with ITF 3756 once a day+anti-CTLA4 continue to grow up to day 48, whereas those of the animals treated with ITF3756 three time a day+anti-CTLA4 remained constant up to day 42 and, then, decreased their volumes.

Unexpectedly, on day 48, 2 out of 5 mice in the group treated with ITF3756 once a day+anti-CTLA4 and 2 out of 4 mice in the group treated with ITF3756 three time a day+anti-CTLA4, were free from tumors.

Moreover, the mice of the two drug combination groups (anti-CTLA4+ITF3756 50 mg/Kg once a day and 3 times a day) were submitted to a second tumor injection (tumor challenge) to monitor the growth of the secondary tumors.

The second injection (1×10⁶ CT26 cells/mouse s.c.) was done in the left inguinal region on day 49 and the animals remained untreated up to day 73. As a control of the optimal growth of the CT26 cells used for the second injection, 5 naïve mice were injected on the same day.

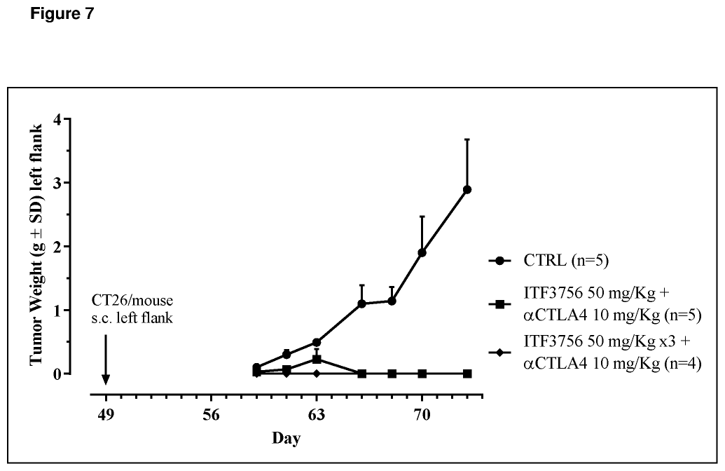

The results obtained are reported in FIG. 7.

The CT26 cells injected in the left flank of naive animals (Control group) showed the expected growth.

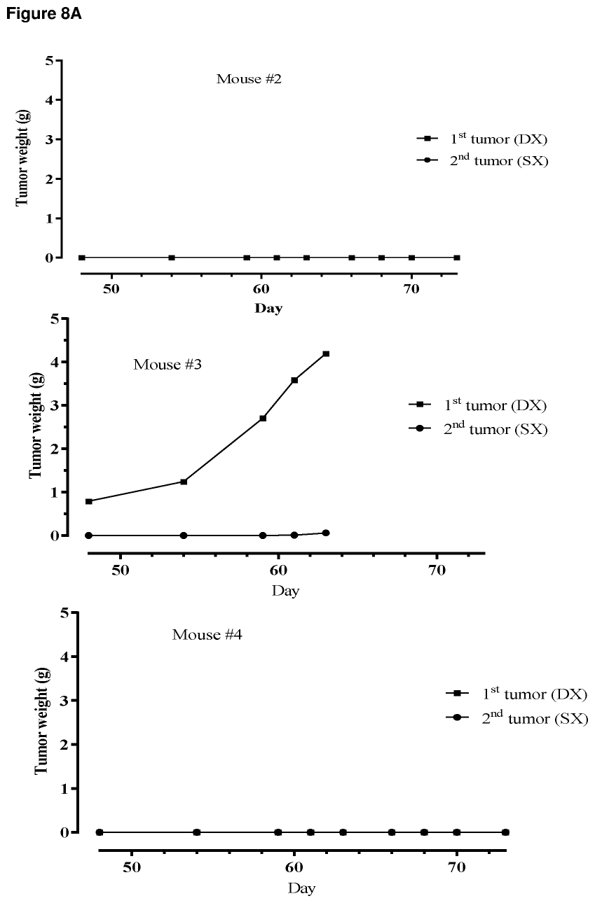

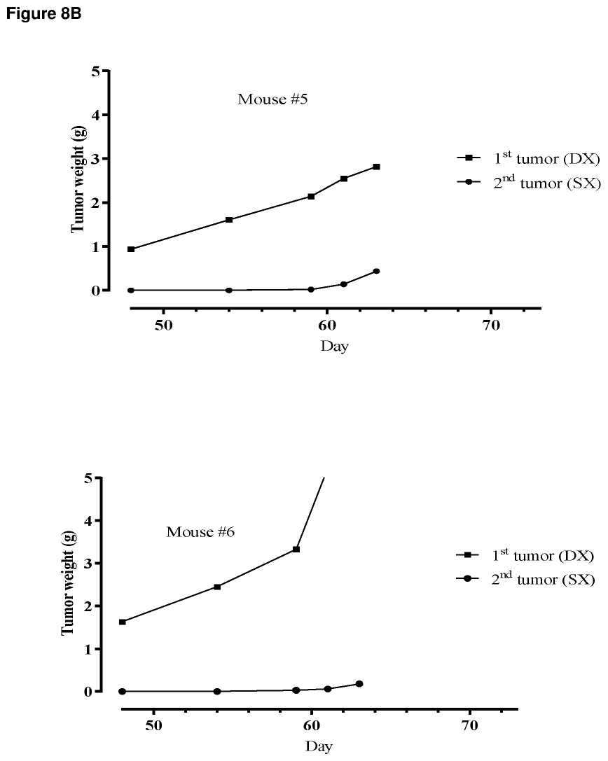

The secondary tumors slightly grow (at day 63) in 3 out of 5 mice (#3, 5 and 6) treated with ITF3756 (50 mg/Kg once a day)+anti-CTLA4 (10 mg/kg), as reported in FIG. 8 (A-B).

It should be noted that, in this group (ITF3756 50 mg/Kg once a day+anti-CTLA4 10 mg/kg), the secondary tumors developed only in tumor-bearing animals on day 48, whereas did not grow in tumor free animals (mice #2 and 4).

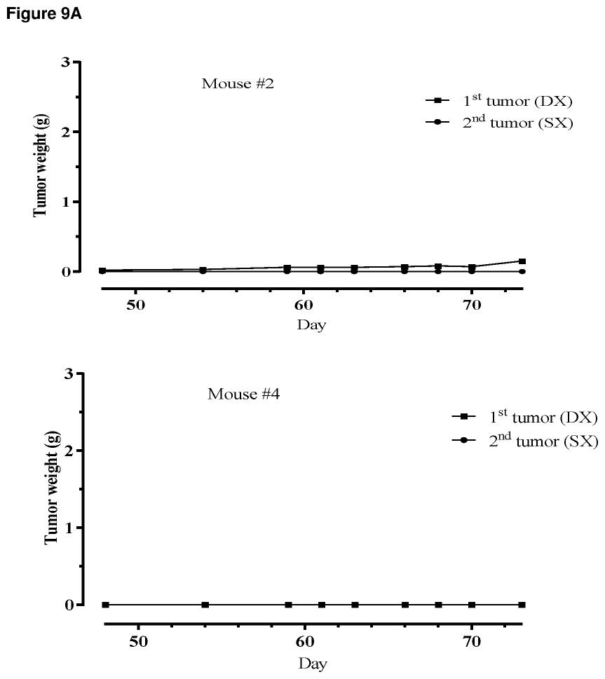

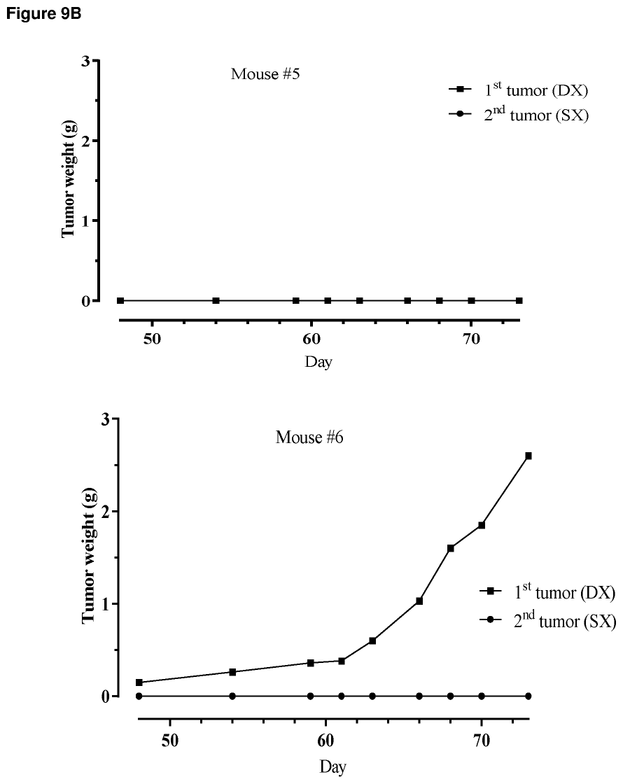

The secondary tumors developed in none of the mice treated with ITF3756 (50 mg/Kg three times a day)+anti-CTLA4 (10 mg/kg), regardless the presence (as in mouse #6) or the absence (as in mice #2, 4 and 5) of primary tumors on day 48, as reported in FIG. 9 (A-B).

### Conclusions

The results obtained may be summarized as follow:
1. ITF3756 administered at 25 and 50 mg/Kg once a day in combination with the anti-CTLA4 antibody shows an anti-tumor effect superior to the administration of the single drugs and this therapeutic effect is synergistic.
2. The most potent anti-tumor effect was obtained with the administration of ITF3756 (50 mg/Kg three times a day) in combination with anti-CTLA4 (10 mg/kg).
3. The combinations of ITF3756 50 mg/Kg once a day or three times a day with anti-CTLA4 10 mg/kg caused a significant delay in tumor growth and, after 73 days of treatment, 2 out of 5 and 4 out of 4 mice were free from tumors, respectively.
4. The secondary tumors did not develop (as in the group treated with ITF3756 50 mg/Kg three times a day+anti-CTLA4 10 mg/kg) or slowly developed in only 3 out of 5 mice (as in the group treated with ITF3756 50 mg/Kg once a day+anti-CTLA4 10 mg/kg). Since both ITF3756 and anti-CTLA4 needs a competent immune system to exert their antitumor effect, this result indicates that an effective antitumor immune response was induced in the treated animals, a response that prevented the growth of the secondary tumor.

### Example 2

Effect of ITF3756 in Combination with Anti CTLA4 to Prevent Tumor Growth Vs. The Combination Anti Pd-1+Anti CTLA4

Balb/c female 6 week-old were submitted to CT26.WT tumor cells injection in the right flank. After about 10 days, whenever tumors were detectable, the treatments started according to the following scheme:
1) ITF3756 50 mg/kg, per os, three times a day (Q3×5)+αCTLA4 10 mg/kg ip 4 times on alternate days, followed by 6 days of washout (Invention);
2) αPD1 3 mg/kg, ip, eod+αCTLA4 10 mg/kg ip 4 times on alternate days followed by 6 days of washout (Reference).

Whenever tumor nodules are detectable in at least 80-85% of the animals, treatments start, and the experiment stops when control group tumor weights are equal to 10% of the animal body weight or (human endpoint) some nodule is ulcerated. Animals were weighted twice a week and tumors measured three times a week.

The measure of the tumor nodule is calculated from the following formula: Volume (mm³)=(D× d²)/2, where D=major diameter of the nodule and d=minor diameter of the nodule.

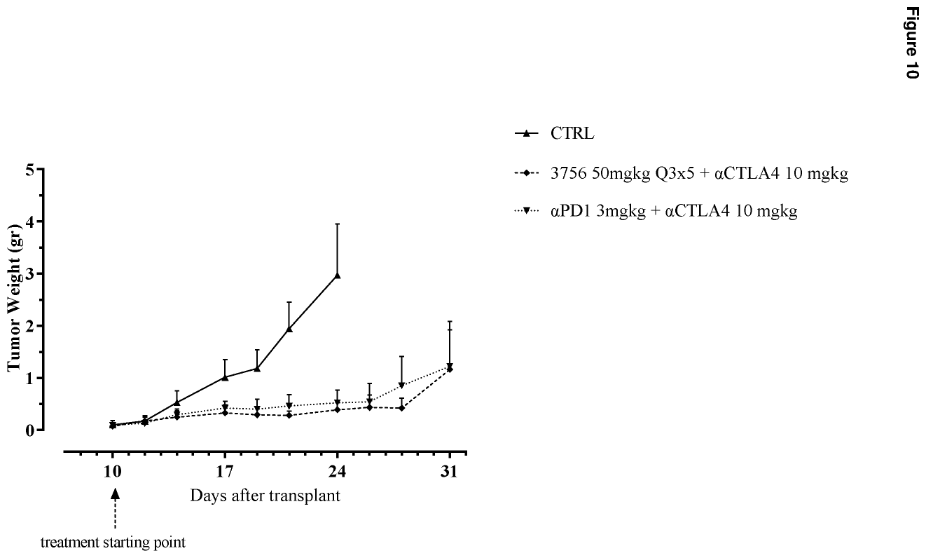

The results reported in FIG. 10 show that there are no statistical differences between the two combo treatments on tumor growth inhibition.

The combination ITF3756 plus anti CTLA4 according to the present invention has the same antitumor activity as the approved combination anti PD-1+anti CTLA4 used as reference.

### Example 3

Effect of ITF3756 Alone or in Combination with Anti CTLA4 on Diabetes in Female Nod Mice

NOD mice spontaneously developed diabetes and they represent a widely accepted spontaneous model of Type 1 diabetes mellitus (Pearson et al., 2016, Journal of Autoimmunity 66, 76-88).

The Programmed Death-1 (PD-1) pathway regulates autoimmune diabetes in NOD mice. PD-1 or PD-L1 blockade rapidly, in less than a week, precipitated diabetes in pre-diabetic, 10-week old female mice (Ansari et al., 2003, The Journal of experimental medicine 198, 63-69).

Blockade of PD-1/PD-L1 axis is also detrimental in other autoimmune models. For example, PD-1^{-/-} mice demonstrated both increased incidence and greater severity of Collagene Induced Arthritis (CIA) than wild-type mice (Raptopoulou et al., 2010. Arthritis & Rheumatism 62, 1870-1880). PD-L1 expressed on macrophages protect from CIA and blocking of PD-L1 during collagen induced arthritis resulted in more severe arthritis (Wood et al., 2020, The Journal of Immunology 204, 73.12). In the Experimental Autoimmune Encephalomyelitis (EAE) model of multiple sclerosis, PD-1 plays a critical role and deletion of PD-1 exacerbates the course of the pathology (Zhang and Braun, 2014, International Immunology 26, 407-415).

HDAC6 inhibition can reduce the expression of PD-L1 in a number of cell types subjected to different stimuli thus affecting the PD-1/PD-L1 axis with potential activation of the immune system.

However, HDAC6 inhibition is effective in models of autoimmune pathologies such as CIA and EAE. Furthermore, in our hands, HDAC6 KO mice developed a milder EAE compared to age and sex matched HDAC6 wild type control.

Collectively, these data indicate that HDAC6 inhibition in the context of autoimmune reactions is protective despite the widely described role of HDAC6 inhibitors in downregulating PD-L1 expression.

Considering the effect of HDAC6i on PD-L1, the present inventors asked whether NOD mice treated with ITF3756 could have a rapid induction of diabetes, like in mice treated with anti PD-(L)1 or instead the diabetes induction was not affected.

Treatment of 10-12 week old mice with anti CTLA-4 has no effect on diabetes induction, however, the present inventors predict that an anti PD-(L)1)/anti CTLA-4 co-treatment generate a comparable effect of anti PD-(L)1 alone.

Our hypothesis was that the NOD mice treated with the selective HDAC6 inhibitor ITF3756 alone or in combination with anti CTLA-4 would not modified the course of diabetes or that only a limited effect would be induced.

### Materials and Methods

### ITF3756 (N-hydroxy-4-((5-(thiophen-2-yl)-1H-tetrazol-1-yl)methyl)benzamide)

ITF3756 was synthetized by the Medicinal Chemistry Dept. of Italfarmaco SpA. ITF3756, batch 9, as powder was solubilized in DMSO and stored at −20° C.

| Identification | ITF3756 lot 9 |
|---|---|
| Type of | Solution in the vehicle |
| Formulation | |
| Dose | 5 mg/ml |
| Concentration | |
| Instruction of | Weight an appropriate amount of test compound and |
| Preparation | make a solution at 100 mg/mL in DMSO. Dilute this |
| | solution with H2O/PEG 1:1 to reach the required |
| | dosage of test article (5 mg/ml solution). Keep under |
| | magnetic stirring. |
| Frequency of | Test formulations was prepared the day of dosing |
| Preparation | |
| Storage | Room temperature |
| Conditions | |
| Source and | Italfarmaco SpA |
| Manufacturer | |
| Method and | Oral, by gastric gavage, using a metal gastric gavage |
| Route | and a graduated plastic syringe. |
| Duration | 5 consecutive days x 2 cycles (Cycle 1: Day 0-4; Cycle |
| | 2: Day 6-8, 11) |
| Frequency | 3 x day 4 hours apart |
| Volume | 10 mL/kg. Individual dose volume was calculated based |
| | on the body weight recorded on the day of dosing. |

### Antibodies

Antibody treatment was according to Ansari (Ansari et al., 2003, The Journal of experimental medicine 1 98, 63-69)

| Identification | Anti mouse PD-1 |
|---|---|
| Lot/Batch Number | 735019O1 and 786520N1B |
| Type of Formulation | Pharmaceutical preparation |
| Dose Concentrations | 500 μg (day 0)/200 μL; 250 μg (day 2, 4, 6, 8, |
| | 11)/200 μL |
| Instruction of Preparation | Ready to use solution at 7.7 mg/mL (lot. |
| | 735019O1) or 8.31 mg/mL (lot. 786520N1B) Then |
| | diluted in PBS at the target concentrations |
| Frequency of Preparation | Test formulations were prepared the day of dosing |
| Storage Conditions | +4° C., protected from light |
| Source and Manufacturer | BioXCell |
| Identification | Anti mouse PD-L1 |
| Lot/Batch Number | 720620A2 |
| Type of Formulation | Pharmaceutical preparation |
| Dose Concentrations | 500 μg (day 0)/200 μL; 250 μg (day 2, 4, 6, 8, |
| | 11)/200 μL |
| Instruction of Preparation | Ready to use solution at 8.29 mg/mL. Then diluted |
| | in PBS at the target concentrations |
| Frequency of Preparation | Test formulations were prepared the day of dosing |
| Storage Conditions | +4° C., protected from light |
| Source and Manufacturer | BioXCell |
| Identification | Anti mouse CTLA-4 |
| Lot/Batch Number | 755620A2 |
| Type of Formulation | Pharmaceutical preparation |
| Dose Concentrations | 500 μg (day 0)/200 μL; 250 μg (day 2, 4, 6, 8, |
| | 11)/200 μL |
| Instruction of Preparation | Ready to use solution at 8.35 mg/mL. Then diluted |
| | in PBS at the target concentrations |
| Frequency of Preparation | Test formulations were prepared the day of dosing |
| Storage Conditions | +4° C., protected from light |
| Source and Manufacturer | BioXCell |
| Identification | Rat IgG2b Isotype Control |
| Lot/Batch Number | 707119D1 |
| Type of Formulation | Pharmaceutical Preparation |
| Dose Concentrations | 500 μg (day 0)/200 μL; 250 μg (day 2, 4, 6, 8, |
| | 11)/200 μL |
| Instruction of Preparation | Ready to use solution at 9.16 mg/mL. Then diluted |
| | in PBS at the target concentrations |
| Frequency of Preparation | Test formulations were prepared the day of dosing |
| Storage Conditions | +4° C., protected from light |
| Source and Manufacturer | BioXCell |

Animals

| Species/Strain or Breed | NOD (non-obese diabetic) Female Mouse |
|---|---|
| Source | Charles River Laboratories, Italia S.p.A. |
| | Via Indipendenza 11, Calco (Italy) |
| Age | Approx. 11-12 weeks at the start of the |
| | treatment period (prediabetic phase of female |
| | nonobese diabetic mice) |
| Weight | Range: 19-27 grams on Day 0 |
| Acclimation | At least 4 days |
| Selection Criteria | Animals were randomized on the basis of |
| | weeks of age, body weight and clinical |
| | observations |
| Identification | Numbered metal tags on the right ear |
| Animals in the study | 90 females |
| Naïve to experimental | Yes |
| procedure status | |
| Animal fate | Animals were sacrificed at the end of the study |

| | | | | | | | Number |
|---|---|---|---|---|---|---|---|
| Test | | | | | | | of |
| Group | Test Item | Item Dose | Route | Volume | | Frequency | Animals |
| 1 | Control/Näive | — | — | — | | — | 10 |
| 2 | Vehicle ^{b} + | 0 | OS | 10 | mL/kg | 3x/day^{a} (Day | 10 |
| | isotype | 500(d0) + | IP | 200 | μL/mouse | 0-4, 6-8, 11)^{c} | |
| | control | 250 μg/mouse | | | | Day 0, 2, 4, | |
| | | | | | | 6, 8, 11 | |
| 3 | ITF3756 | 50 mg/kg | OS | 10 | mL/kg | 3x/day^{a} (Day | 10 |
| | | | | | | 0-4, 6-8, 11)^{c} | |
| 4 | Anti-PD-1 | 500(d0) + | IP | 200 | μL/mouse | Day 0, 2, 4, | 10 |
| | | 250 μg/mouse | | | | 6, 8, 11 | |
| 5 | Anti-PD-L1 | 500(d0) + | IP | 200 | μL/mouse | Day 0, 2, 4, | 10 |
| | | 250 μg/mouse | | | | 6, 8, 11 | |
| 6 | Anti-CTLA-4 | 500(d0) + | IP | 200 | μL/mouse | Day 0, 2, 4, | 10 |
| | | 250 μg/mouse | | | | 6, 8, 11 | |
| 7 | ITF3756 + | 50 mg/kg | OS | 10 | mL/kg | 3x/day^{a} (Day | 10 |
| | anti-CTLA-4 | 500(d0) + | IP | IP | | 0-4, 6-8, 11)^{c} | |
| | | 250 μg/mouse | | | | Day 0, 2, 4, | |
| | | | | | | 6, 8, 11 | |
| 8 | Anti-PD1 + | 500(d0) + | IP | 200 | μL/mouse | Day 0, 2, 4, | 10 |
| | anti-CTLA-4 | 250 μg/mouse | IP | 200 | μL/mouse | 6, 8, 11 | |
| 9 | Anti-PD-L1 + | 500(d0) + | IP | 200 | μL/mouse | Day 0, 2, 4, | 10 |
| | anti-CTLA-4 | 250 μg/mouse | IP | 200 | μL/mouse | 6, 8, 11 | |

^{a}= second and third daily doses about 4 hours apart from the previous administration
^{b} = PEG 400/H₂0 (1:1) containing DMSO (max 5%)
^{c}= Cycle 1: Day 0-4; Cycle 2: Day 6-8, 11

### Glucose Level Measurements

Glycemia was evaluated in all mice for 3 weeks from day 0 (start treatment) until day 21 (end of study). Blood glucose levels were measured by glucometer One Touch® Verio. Blood samples were drawn from the tail vein by a prick with a needle. A drop of blood was inserted in a test strip and glucometer automatically calculated the glucose level. Results were shown in the display of the instrument. The instrument measures from 20 to 600 mg/dL. Results below 20 or above 600 mg/dL are visualized by the instrument as “below 20” or “above 600”.

Diabetes was defined by blood glucose reading of >250 mg/dL for two consecutive days.

Mice were monitored for 3 weeks; during this time, body weight was measured 7 times.

### Results

Treatments with ICI are endowed with intrinsic possibility to generate autoimmune reactions that has been confirmed by the clinical practice. Combination of two ICI, such as anti CTLA4 and anti PD-1, has a better efficacy with a higher incidence of adverse events. The goal of tumor immunotherapy is therefore to improve efficacy and reducing side effects to a manageable level. The combination of the selective HDAC6 inhibitor ITF3756 with anti CTLA4 has an antitumor efficacy comparable to anti CTLA4+anti PD-1 and our data on NOD mice demonstrate that it has an excellent safety profile.

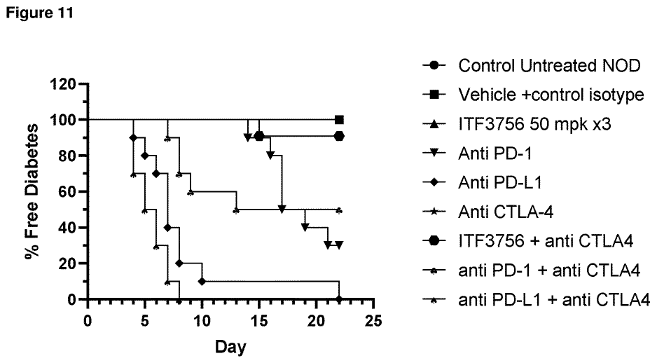

The results obtained in the NOD model show a drastic induction of diabetes in mice treated with anti PD-L1 and the combination (anti PD-L1+anti CTLA-4). These findings are in agreement with literature data and inventors' prediction, respectively (FIG. 11).

The other two groups that showed an increase of diabetes incidence were the anti PD-1 and (anti PD-1+anti CTLA-4) with a diabetes incidence of 60% and 50% at day 21 respectively.

In striking contrast, only one mouse in (ITF3756+anti CTLA-4) group had a transient increase of glucose level between day 11 and 14. However, said mouse partially recovered since all measurements after day 15 were below 250 mg/dL, although higher than the mean value of control group.

No induction of diabetes was detected in the other groups.

The results obtained in the groups treated with the antibodies, are in agreement with the literature data that described a critical role of the PD-1/PD-L1 axis in NOD mice.

The results clearly indicate that HDAC6 inhibition does not increase diabetes incedence and notably that the combination of the selective HDAC6 inhibitor ITF3756 with an anti CTLA-4 antibody has a very marginal effect on diabetes induction.

On the other hand, the combination of anti CTLA-4 antibody with both anti PD-1 and anti PD-L1 antibodies strongly exacerbate diabetes.

This results suggest that the (anti CTLA-4+ITF3756) treatment could have a better safety profile than the anti CTLA-4+anti PD-(L)1.

### Example 4

Anti-Tumor Effect of ITF3756 Administered in Combination with Anti-CTLA4 Antibody in the 4T1 Breast Cancer Model

C57BL/6 mice challenged with syngeneic tumor cell line 4T1 triple negative breast cancer are used.

4T1 tumor model is considered a poorly immunogenic tumor (Demaria et al., Clin. Cancer Res. 11, 728-734, 2005). According to previous experiments, anti CTLA-4 treatment of this tumor shows general efficacy but variability among the treated animals and therefore 4T1 constitutes a good model to test immunotherapy-based combinations therapies.

ITF3756 is used at the same dosage used for the treatment of CT26 colon cancer, namely 50 mg/kg (mpk) TID as single agent and in combination with anti CTLA-4 antibody. The latter is administered at 3 mpk 3 times a week on alternate days. In the previous experiments, the present inventors observed a comparable average reduction of tumor growth of approximately 40% for both drugs. Anti CTLA-4 gave comparable tumor reduction at 3 mpk and 10 mpk while the activity at 1 mpk was marginal.

Tumor growth is induced after subcutaneous injection of cell line and mice are monitored for at least three weeks post-injection.

### Example 5

Anti-Tumor Effect of ITF3756 Administered in Combination with Anti-CTLA4 Antibody in the B16F10 Melanoma Model

C57BL/6 mice challenged with syngeneic tumor cell line B16F10 melanoma are used.

The B16F10 melanoma model is also considered a poorly immunogenic tumor (Reilley et al., J. Immunother. Cancer 7, 323, 2019) and therefore represent another good model to test immunotherapy-based combinations.

ITF3756 is used at the same dosage used for the treatment of CT26 colon cancer, namely 50 mg/kg (mpk) TID as single agent and in combination with anti CTLA-4 antibody.

Regarding the dosage of the anti CTLA-4 antibody in this model, the present inventors performed a preliminary experiment to ascertain the optimal dose that can be combined with ITF3756. According to the literature, the anti CTLA-4 antibody is administered at different dosages, the dose 200 ug/mouse at day 3, followed by 100 ug/mouse at day 6, 9 and 12 after tumor cell injection, (Reilley et al., 2019; Sharma et al., Proc. Natl. Acad. Sci. U.S.A. 116, 10453-10462, 2019) was used as a guide for this experiment.

Tumor growth is induced after subcutaneous injection of cell line and mice are monitored for at least three weeks post-injection.

1-11. (canceled)

12. A combination comprising N-hydroxy-4-((5-(thiophen-2-yl)-1H-tetrazol-1-yl)methyl)benzamide, or a pharmaceutically acceptable salt thereof, and at least one CTLA4 checkpoint inhibitor.

13. The combination according to claim 12, wherein the at least one CTLA4 checkpoint inhibitor is ipilimumab or tremelimumab.

14. A method of treating a disease or condition susceptible to being improved or prevented by treatment with anti CTLA4, anti PD1 and/or anti PD-L1 antibodies in a subject in need thereof, comprising administration of an effective amount of the combination according to claim 12.

15. The method according to claim 14, wherein the subject has discontinued treatment with anti CTLA4, anti PD1 and/or anti PDL1 antibodies.

16. The method according to claim 14, which method comprises immunotherapy of tumors and/or treatment of one or more HDAC6-mediated diseases.

17. The method according to claim 16, wherein the subject is not treated with anti CTLA4, anti PD1 and/or anti PDL1 antibodies.

18. The method according to claim 16, which method comprises treatment of one or more diseases selected from the group consisting of: melanoma, breast cancer, renal cell carcinoma, non small cell lung cancer and colorectal cancer.

19. The method according to claim 17, which method comprises treatment of one or more diseases selected from the group consisting of: melanoma, breast cancer, renal cell carcinoma, non small cell lung cancer and colorectal cancer.

20. The method according to claim 14, wherein the N-hydroxy-4-((5-(thiophen-2-yl)-1H-tetrazol-1-yl)methyl)benzamide or a pharmaceutically acceptable salt thereof and the at least one CTLA4 checkpoint inhibitor are administered simultaneously, separately or sequentially.

21. The method according to claim 20, wherein the N-hydroxy-4-((5-(thiophen-2-yl)-1H-tetrazol-1-yl)methyl)benzamide is administered on a daily basis, and the at least one CTLA4 checkpoint inhibitor is administered every 2 to 4 weeks.

22. The method according to claim 21, wherein the N-hydroxy-4-((5-(thiophen-2-yl)-1H-tetrazol-1-yl)methyl)benzamide is administered from two to three times a day.

23. The method according to claim 21, wherein the N-hydroxy-4-((5-(thiophen-2-yl)-1H-tetrazol-1-yl)methyl)benzamide is administered orally.

24. The method according to claim 21, wherein the at least one CTLA4 checkpoint inhibitor is administered by intravenous infusion.

25. The method according to claim 21, wherein the N-hydroxy-4-((5-(thiophen-2-yl)-1H-tetrazol-1-yl)methyl)benzamide is administered in an amount ranging from 200 mg to 1000 mg BID or from 100 mg to 1000 mg TID and the at least one CTLA4 checkpoint inhibitor is administered in an amount ranging from 0.5 to 10 mg/kg every 2 to 4 weeks.

26. The method according to claim 25, wherein the CTLA4 checkpoint inhibitor is administered in an amount ranging from 1 to 3 mg/kg every 2 to 4 weeks.
